SEQUENCE LISTING (4 sequences)

SEQ ID NO: 1 - DNA, 22 bp, Artificial Sequence
  Primer
actgtagtgagagatgaagagg

SEQ ID NO: 2 - DNA, 20 bp, Artificial Sequence
  Primer
ggcgtatacctgttcataat

SEQ ID NO: 3 - DNA, 2074 bp, Canis lupus familiaris
actgtagtgagagatgaagagggacactatatcatacttaaaggatctatccaacaagaggacttaacaatcctcaatatatatgccccgaatgtgggagctgccaaatatataaatcaattattaaccaaagtgaagaaatacttagataataatacacttatacttggtgacttcaatctagctctttctatactcgataggtcttctaagcacaacatctccaaagaaacgagagctttaaatgatacactggaccagatggatttcacagatatctacagaactttacatccaaactcaactgaatacacattcttctcaagtgcacatggaactttctccagaatagaccacatattgggtcaccaatcgggtctgaaccgataccaaaagattgggatcgtcccctgcatattctcagaccataatgccttgaaattagaactaaatcacaacaagaagtttggaaggacctcaaacacgtggaggttaaggaccatcctgctaaaagatgaaagggtcaaccaggaaattaaggaagaattaaaaagattcatggaaactaatgagaatgaagatacaaccgttcaaaatctttgggatgcagcaaaagcagtcctgagggggaaatacatcgcaatacaagcatccattcaaaaactggaaagaactcaaatacaaaagctaaccttacacataaaggagctagagaaaaaacagcaaatggatcctacacccaggagaagaagggagttaataaagattcgagcagaactcaacgaaatcgaaaccagaagaactgtggaacagatcaacagaaccaggagttggttctttgaaagaattaataagatagataaaccattagccagccttcttaaaaagaagagagagaagactcaaattaataaaatcatgaatgagaaaggagagatcactaccaacaccaaggaaatacaaacgattttaaaaacatattatgaacaggtatacgccaataaattaggcaatctagaagaaatggacgcattcctggaaagccacaaactaccaaaactggaacaggaagaaatagaaaacctgcacaggccaataaccagggaggaaattgaagcagtcatcaaaaacctcccaagacacaagagtccagggccagatggcttcccaggggaattttatcaaacgtttaaagaagaaatcatacctattctcctaaagctgtttggaaagatagaaagagatggagtacttccaaattcgttttatgaagccagcatcaccttaattccaaaaccagacaaagaccccaccaaaaaggagaattacagaccaatatccctgatgaacatggatgcaaaaattctcaacaagatactggccaataggatccaacagtacattaagaaaattattcaccatgaccaagtaggatttatccccgggacacaaggctggttcaacacccgtaaaacaatcaatgtgattcatcatatcagcaagagaaaaaccaagaaccatatgatcctctcattagatgcagagaaagcatttgacaaaatacagcatccattcctgatcaaaactcttcagagtgtagggatagagggaacattcctcgacatcttaaaagccatctacgaaaagcccacagcaaatatcattctcaatggggaagcactgggagcctttcccctaagatcaggaacaagacagggatgtccactctcaccactgctattcaacatagtggtggaagtcctagcctcagcaatcagacaacaaaaagactttaggggcattcaatttggcaaagaagaagtcaaactctccctcttcgccgatgagatgatcctctacatagaaaacccaaaagtctccaccccaagattgctacaactcatgcagcattgtggtagcgtggcaggatacatcatcaatgcccagaaatcagtggcatttctatacactaacaatgagactgaagaaagagaaattaaggagtcaatcccatttacaattgcacccaaaagcataagatacctaggaataaacctaaccagggaggtaaagg

SEQ ID NO: 4 - DNA, 29 bp, Canis lupus familiaris
aaaaatacatacatacatacatacataca

(19) United States
(12) Patent Application Publication
(10) Pub. No.: US 2015/0161359 A1
(43) Pub. Date: Jun. 11, 2015

(54) METHODS FOR SAFETY TESTING

(71) Applicant: NOVARTIS AG, Basel (CH)

(72) Inventors: Heidi Trusheim, Apex, NC (US); Linda Rathaj Hammer, Marburg (DE); Reinhard Wilms, Marburg (DE); Walter Hoyer, Marburg (DE)

(21) Appl. No.: 14/403,496

(22) PCT Filed: May 31, 2013
(86) PCT No.: PCT/EP2013/061331

§ 371 Date: Nov. 24, 2014

Related U.S. Application Data
(60) Provisional application No. 61/655,178, filed on Jun. 4, 2012.

(51) Int. Cl. G06F 19/00; C12Q 1/70

(52) U.S. Cl. CPC G06F 19/706; C12Q 1/701; C12Q 2600/158

(57) ABSTRACT

The invention provides improved methods for the safety testing of compositions which comprise biological products produced in a host cell, such as vaccine antigens or recombinant proteins.

This patent application claims priority from U.S. provisional patent application 61/655,178, filed Jun. 4, 2012, the complete contents of which are incorporated herein by reference.

This invention was made in part with Government support under grant no. HHSO100200600012C. The Government has certain rights in the invention.

### TECHNICAL FIELD

This invention is in the field of safety testing for biological products.

### BACKGROUND ART

To characterize the risk of residual DNA in cell derived vaccines, a DNA safety factor can be determined. The safety factor indicates the number of vaccine doses that an individual must be injected with in order to trigger a tumorigenic event with a probability of at least 5%. In 2005, the US Food and Drug Administration (FDA) recommended a safety factor of >10⁷ for vaccines with antigens that were prepared in mammalian cell culture [1].

Previously the safety of cell culture based biological products was assessed by determining the residual DNA size distribution by capillary gel electrophoresis. By assessing the number of DNA fragments with a defined length, the potential number of oncogenes per human dose and the resulting DNA safety factor were calculated. However, this method does not exclude non-functional long DNA fragments (e.g. alkylated or nicked DNA) from the calculation and so overestimates the actual number of functional oncogenes in a sample.

Reference 2 discloses a method for determining the safety factor by relating an estimated number of oncogenes to the total amount of DNA in the sample. It teaches that the safety factor is often inaccurate due to enzyme inactivation and suggests alternative calculations for taking enzyme inactivation into account.

It is an object of the invention to provide further and more accurate assays for assessing the safety of cell culture derived biological products, such as vaccines.

### DISCLOSURE OF THE INVENTION

The inventors have now provided improved assays for assessing the safety of cell culture derived biological products which allow the DNA safety factor to be assessed more accurately.

In one embodiment, the invention provides a method for determining the DNA safety factor of a composition comprising a biological product produced in a host cell, comprising the steps of:
a) amplifying at least a fragment of a repetitive element or of a housekeeping gene of the host cell;
b) using the amplified DNA to determine the copy number of the repetitive element or the housekeeping gene;
c) using the copy number of the repetitive element or the housekeeping gene to calculate the number of oncogenes in a dose of the composition N_{dose}^{genes}; and
d) determining the DNA safety factor (SF).

The DNA safety factor SF is preferably determined by the formula

$$SF = \frac{{N}_{critical}^{oncogenes}}{{N}_{dose}^{oncogenes}}$$

in which N_{critical}^{oncogenes} is the maximum number of oncogenes per dose which may be present in a dose of the composition.

Due to the amplification step, non-functional DNA fragments which are, for example, nicked or alkylated are excluded from the analysis because such DNA molecules are poor templates for amplification. Accordingly, only functional DNA will be taken into account when calculating the DNA safety factor which improves the accuracy of the method.

The invention further provides a composition comprising a biological product produced in a host cell, wherein the composition comprises fewer than n repetitive elements or housekeeping genes per mL, wherein n is calculated by the formula

$$n = \frac{320}{R} \frac{oncogenes}{mL}$$

in which R=ratio of oncogenes to repetitive elements/housekeeping genes in the host cell.

Also provided is a method for making a composition comprising a biological product, comprising the steps of (a) culturing a host cell to produce the biological product; (b) preparing a composition from the biological product produced in (a); and (c) determining the DNA safety factor of the composition by a method according to the invention. The composition may be a vaccine composition and the method may comprise the steps of (a) culturing a host cell to produce a virus; (b) preparing a vaccine from the virus produced in (a); and (c) determining the DNA safety factor of the vaccine composition by a method according to the invention.

The invention further provides a method of characterizing a cell, comprising the steps of
a) amplifying at least a fragment of a repetitive element or a housekeeping gene of the cell;
b) using the amplified DNA to determine the copy number of the repetitive element or the housekeeping gene in the cell; and
c) calculating the ratio R of oncogenes to repetitive element/housekeeping gene.

The ratio R is preferably calculated by the formula

$$R = \frac{{N}_{onco} \times {c}_{DNA}}{{m}_{hap . Gen} \times {N}_{rep / mL}}$$

with
   N_{onco}: number of oncogenes per genome
   Nᵣₑₚ: number of repetitive elements/housekeeping genes [rep/mL]
   c_{DNA}: concentration of the cell DNA in the test sample [pg/mL]
   M_{hap.gen}: mass of the haploid genome of the cell.

### Safety Factor

The safety factor (SF) of a vaccine is calculated by the formula

$$SF = \frac{{N}_{critical}^{oncogenes}}{{N}_{dose}^{oncogenes}}$$

in which N_{critical}^{oncogenes} is the maximum number of oncogenes which can safely be present in a dose of the composition and N_{dose}^{oncogenes} is the calculated number of oncogenes per dose of the composition.

The critical dose of oncogenic DNA to induce a tumour was determined by the US Food and Drug Administration (FDA). It was found that an amount of 800 pg of a linear DNA fragment which encodes two oncogenes was enough to cause tumours in test animals. This amount is equivalent to 8.0×10⁷ dual oncogenes based on the length of the expression plasmid used in the study [3]. The design of the construct, where both genes were located on the same DNA fragment, has been shown previously to be more efficient at inducing tumours than if the oncogenes are on separate plasmids. The gain in efficiency was approximately a factor of 20, i.e. approximately 20-fold less total DNA was needed to induce tumours when the two oncogenes were located on the same plasmid than when they were on separate plasmids. Based on this information the critical number of oncogene fragments needed to induce tumours can be calculated by the formula

N_{critical}^{oncogenes}=8.0·10⁷×20=1.6·10⁹

In order to assess the DNA safety factor based on the number of repetitive elements and/or housekeeping genes in a composition it is also necessary to know the ratio between oncogenes and the repetitive element/housekeeping gene in the host cell's genome. This ratio can be determined, for example, by extracting genomic DNA from the host cell in which the biological product is to be produced, optionally diluting the extracted DNA, and providing a dilution series of the extracted DNA. The diluted samples are subjected to DNA amplification (for example by PCR) using primers which amplify at least a fragment of the repetitive element or the housekeeping gene. The samples are then analysed, for example, by agarose gel electrophoresis and the highest dilution at which no amplification product is detectable is determined. The copy number of the repetitive element or the housekeeping gene (Nᵣₑₚ) can be determined by the following formula in which ‘DL’ is the detection limit of the specific repetitive element or housekeeping gene under the given conditions (repetitive element/mL), ‘PCR(−)’ indicates the dilution factor of the first negative amplification signal and ‘dilution’ refers to the dilution factor of any initial dilution which may optionally have taken place after DNA extraction:

Nᵣₑₚ/mL=DL×PCR(−)×dilution

The detection limit DL for the specific repetitive element or housekeeping gene will vary with the amplification conditions used. It can be determined by cloning the fragment of the repetitive element or housekeeping gene which is to be amplified in the assay into an expression vector and preparing a dilution series of the vector with known amounts of the plasmid. The vector is used as a template for DNA amplification using the specific amplification conditions which are intended to be used in the assay and the lowest detectable amount of vector under these conditions is determined, for example by determining the dilution factor at which no amplification product can be detected on an agarose gel. In order to calculate the copy number, the number of nucleotides in the vector is determined and multiplied with a factor of 1.1×10⁻⁹ pg/bp. From this calculation one can determine the copy numbers of the vector by dividing the weight of the total amplified DNA by the weight of the individual vector. As each vector contains only a single fragment, the number of vectors in the lowest dilution in which amplification products are still detectable is equivalent to the detection limit of the fragment. Using this technique, the inventors were able to demonstrate, for example, that the detection limit of LINE elements is 10.000 LINES per mL in Tris Buffer.

The ratio of oncogenes to repetitive element/housekeeping gene R can be calculated as follows:

$$R = \frac{{N}_{onco}}{{N}_{rep}} = \frac{{N}_{onco} \times {c}_{DNA}}{{m}_{hap . Gen} \times {N}_{rep / mL}}$$

with
   N_{onco}: number of oncogenes per genome
   Nᵣₑₚ: number of repetitive elements/housekeeping genes [rep/mL]
   c_{DNA}: concentration of the host cell DNA in the test sample [pg/mL]
   M_{hap.gen}: mass of the haploid genome

The mass of the haploid genome of the host cell will usually be known in the art and can be found, for example, in the Animal Genome Size Database[4]. For example, the haploid genome of MDCK cells has a mass of 3.09 pg per haploid genome and CHO cells are estimated to have a mass of 2.73 pg per haploid genome. Likewise, the number of oncogenes in a genome can be derived from the literature. For example, MDCK cells are estimated to have 10 oncogenes per genome.

Using these values, the inventors have calculated, for example, that the value for LINE elements in MDCK cells is 6.2×10⁻⁶ oncogenes per LINE element.

The ratio R can be determined for each host cell individually. However, biological products are often grown in specific cell lines which are suitable for the production of pharmaceutical compositions. Examples of such cell lines include MDCK cells (like MDCK 33016 [33]), CHO cells, Vero cells (e.g. those obtainable under catalogue numbers CCL 81, CCL 81.2, CRL 1586 and CRL-1587 American Type Cell Culture (ATCC) collection), 293T cells and PER.C6 cells. These cells can be obtained from a working or master cell bank (such as those available from the American Type Cell Culture (ATCC) collection[5], from the Coriell Cell Repositories [6], or from the European Collection of Cell Cultures (ECACC)) and it is possible to determine the ratio R in cells from such cell banks. This is preferred because it avoids the need to test each host cell individually. The invention therefore provides a method of characterizing a cell, comprising the steps of (a) amplifying at least a fragment of a repetitive element or a housekeeping gene of the cell; (b) using the amplified DNA to determine the copy number of the repetitive element or the housekeeping gene in the cell; and calculating the ratio of R by the formula discussed above.

Host cells which have been characterised by the methods of the invention can be used in methods for making a biological product. The invention thus provides a method for making a biological product comprising the steps of (a) characterising a host cell by a method of the invention; and (b) using a culture of the host cell to prepare a biological product. The biological product may be a virus in which case the method may comprise the steps of (a) characterizing a host cell by a method of the invention; and (b) using a culture of the host cell to prepare a virus (e.g. by infecting the host cell with a virus or transfecting it with one or more expression construct(s) for reverse genetics). The step of preparing the virus may involve the steps of infecting the host cell culture with a virus or transfecting it with one or more expression construct(s) for reverse genetics, and culturing the host cell culture to produce the virus. It will be understood that the step of infecting the host cell culture with a virus does not require that every single cell in the culture is infected. Instead, it is sufficient if one or more cell(s) in the culture is/are infected.

Also provided is a method for making a composition comprising the steps of (a) characterising a host cell by a method of the invention; (b) using a culture of the host cell to prepare a biological product; and (c) preparing a composition which comprises (i) the biological product produced in step (b) or (ii) a compound made from the biological product produced in step (b). Where the composition is a vaccine, the method may comprise the steps of (a) characterizing a host cell by a method of the invention; (b) using a culture of the host cell to produce a virus (e.g. by infecting the host cell with a virus or transfecting it with one or more expression construct(s) for reverse genetics); (c) culturing the cell to produce a virus; and (d) using the virus produced in (c) to prepare a vaccine.

Where biological products are produced in cells, it is common practice to use a cell-bank system (also known as cell-seed system). These systems are well known in the art. Briefly, these systems involve that the biological product is produced in cells which are derived from a master cell bank (master cell seed). From the master cell bank, one or more working cell banks (working cell seeds) can be produced (for example by dilution, passaging etc.). The methods of the invention can be used to characterise a cell from a master cell bank or any cell bank derived from a particular master cell bank. The invention thus provides a method for making a biological product, comprising the steps of (a) characterising a cell from a cell bank by a method of the invention or providing a cell from a cell bank which has been characterised by a method of the invention; and (b) using a cell from the cell bank to prepare a biological product. The biological product may be a virus in which case the method comprises the steps of (a) characterising a cell from a cell bank by a method of the invention or providing a cell from a cell bank which has been characterised by a method of the invention and (b) using a cell from the cell bank to prepare a virus. The step of preparing the virus may involve the steps of infecting the cell with a virus and culturing the cell to produce the virus. The cell which is characterised in step (a) of these methods may be a cell from the same cell bank which is used to produce the biological product in step (b). The cell may also be from a different cell bank provided that the cell which is characterised and the cell which is used to produce the biological product are clones or progeny of the same master cell bank.

Also provided is a method of making a composition, comprising the steps of (a) characterising a cell from a cell bank by a method of the invention or providing a cell from a cell bank which has been characterised by a method of the invention; (b) using a cell from the cell bank to prepare the biological product; and (c) preparing a composition which comprises (i) the biological product produced in step (b) or (ii) a compound made from the biological product produced in step (b). Where the composition is a vaccine, the method may comprise the steps of (a) characterising a cell from a cell bank by a method of the invention or providing a cell from a cell bank which has been characterised by a method of the invention; (b) using a cell from the cell bank to prepare a virus; and (c) preparing a vaccine from the virus produced in (b).

The cell used in step (b) may be a progeny of the cell used in step (a) and/or it may be a clone of the cell used in step (a) and/or it may be a cell which has been passaged from the cell used in step (a) at least once between steps (a) and (b). The method of characterising a cell can also be used in a method for preparing a vaccine in which (a) a cell is characterised in accordance with a method of the invention; (b) the cell is infected with a virus; (c) the cell is cultured to produce a virus; and (d) a vaccine is prepared from the virus produced in (c).

The cell used in steps (a) and (b) of the methods of the preceding paragraphs will not usually be identical as, for example, the step of characterising the host cell will usually damage or destroy the cell. Thus, the cell used in step (b) may be a progeny of the cell used in step (a) and/or it may be a clone of the cell used in step (a) and/or it may be a cell which has been passaged from the cell used in step (a) at least once between steps (a) and (b). The cell may have been passaged at least two times (for example more than 5 times, more than 10 times, more than 20 times or more than 40 times) between steps (a) and (b). The cells used in these methods may be from a cell line like, for example, MDCK, CHO, Per.C6 or Vero cells.

Once the ratio R is known, the concentration of oncogenes in the composition (c_{onco}) can be calculated by the formula:

c_{onco}=PCR(−)×DL×R

in which ‘DL’ is the detection limit of the specific repetitive element or housekeeping gene under the given conditions (repetitive element/mL) and ‘PCR(−)’ indicates the dilution factor of the first negative PCR signal.

Instead of PCR it is also possible to practise the invention using any known DNA amplification technique. The described methods and calculations will then be adapted to the specific amplification technique used. For example, when the DNA is amplified using rolling-circle (RCL) amplification the above calculation will be adapted to refer to the dilution factor of the first negative RCL signal.

The actual number of oncogenes in a dose (N_{dose}^{oncogenes}) is a theoretical consideration which can be calculated depending on different factors. For example, the DNA safety factor can be calculated using the assumption that a dose of the composition has a DNA content of x ng, for example 10 ng. In this case the safety factor is projected to the assumed DNA content of a dose of the composition (for example 10 ng) and is calculated by the formula

$$SF = \frac{{N}_{critical}^{oncogenes}}{{c}_{onco} \times \frac{10 ng}{{c}_{DNA}}}$$

in which c_{onco} is the concentration of oncogenes in the monobulk [oncogenes/mL] and c_{DNA} is the DNA concentration in the monobulk [ng/mL]. The DNA content can be measured by quantitative DNA assays, for example, by a Threshold™ assay which is a quantitative assay for picogram levels of total DNA that has been used for monitoring levels of contaminating DNA in biopharmaceuticals [7]. A typical assay involves non-sequence-specific formation of a reaction complex between a biotinylated ssDNA binding protein, a labelled (e.g. a urease-conjugated) anti-ssDNA antibody, and DNA. Alternative ways of quantitating DNA are also known in the art and include hybridization methods, such as Southern blots or slot blots [8] and quantitative PCR [9]. Various commercial manufacturers offer quantitative PCR assays for detecting residual host cell DNA e.g. AppTec™ Laboratory Services, BioReliance™, Althea Technologies, etc. A comparison of a chemiluminescent hybridisation assay and the total DNA Threshold™ system for measuring host cell DNA contamination of a human viral vaccine can be found in reference 10.

Alternatively, the safety factor SF may be calculated in respect of the dose volume of the composition. In this embodiment, the DNA safety factor is calculated by the formula

$$SF = \frac{{N}_{critical}^{oncogenes}}{{c}_{onco} \times {V}_{dose}}$$

in which V_{dose} is the volume of a dose of the composition [mL].

Calculating the safety factor SF in relation to the dose volume (as opposed to the DNA content) has the advantage that this provides a more realistic value of the safety factor because a dose of a composition frequently contains significantly less than 10 ng of DNA and so extrapolating the safety factor to this DNA content may mean that the safety factor is calculated for a theoretical dose which significantly exceeds the actual dose volume of the composition.

The most realistic assessment of the DNA safety factor is to calculate it in respect of the concentration of the biological product per dose (c_{dose}), for example the antigen content of a vaccine or the amount of the recombinant proteins. The safety factor is calculated by the formula

$$SF = \frac{{N}_{critical}^{oncogenes}}{{c}_{onco} \times \frac{{c}_{dose}}{{c}_{actual}}}$$

in which c_{dose} is the concentration of the biological product per dose [μg/dose] and c_{actual} is the actual concentration of the active ingredient in the composition [μg/mL]. For example, a trivalent seasonal influenza vaccine typically has a hemagglutinin (HA) concentration of 45 μg HA per dose in which the case the safety factor is calculated by the formula:

$$SF = \frac{{N}_{critical}^{oncogenes}}{{c}_{onco} \times \frac{45 \frac{µg}{dose}}{{c}_{actual}}}$$

Methods of measuring the actual concentration of the active ingredient in the sample are known in the art. For example, the HA content of vaccines can be determined by single radial immunodiffusion (“SRiD”) assays [11,12] and the total protein concentration can be measured by the Bradford assay [13].

Compositions which have a safety factor of less than 10⁷ may be rejected.

### The Composition

Compositions of the invention comprise a biological product which can be any product that can be produced in a host cell. Suitable biological products include vaccine antigens derived from viruses grown in cell culture and recombinant proteins.

Compositions of the invention can comprise a single biological product but can also comprise two or more biological products, for example two or more different antigens or recombinant proteins. The two or more biological products were preferably all produced in a host cell. However, compositions of the invention may also comprise biological products which were not produced in a host cell provided that it comprises at least one biological product which was produced in a host cell.

The composition is preferably free from egg-derived materials (e.g. free from ovalbumin, free from ovomucoid, free from chicken DNA). The biological product in the composition will preferably be glycosylated with glycans obtainable from growth in a mammalian cell line (e.g. the cell lines described herein), such as MDCK.

The composition may be a vaccine composition which comprises a biological product which is an antigen derived from a virus which can be grown in cell culture. Such viruses are known in the art and include, for example, influenza virus, vaccinia virus, poliovirus, Hepatitis A Virus, Hepatitis B virus, Hepatitis C virus, Ross River Virus, Yellow fever virus, West nile virus, Japanese encephalitis virus, rubella virus, mumps virus, measles virus, respiratory syncytial virus, Herpes Simplex Virus, Cytomegalovirus, Epstein-Barr Virus, rotavirus, measles, mumps, rubella, rabies and yellow fever.

The compositions are preferably compositions, in particular vaccine compositions, which are intended to be used in humans. This is preferred because it is of paramount importance in such vaccines that the safety factor is determined accurately. Examples of such vaccines are influenza vaccines (e.g. Optaflu™) rabies vaccines (e.g. RabAvert™), Hepatitis B vaccines (GenHevac B™), measles vaccines (e.g. Attenuvax™), mumps vaccines (e.g. Mumpsvax), rubella vaccines (e.g. Meruvax II™), and measles mumps and rubella combination vaccines (e.g. M-M-R II™).

The invention is particularly suitable for influenza vaccine compositions because the growth of influenza vaccines in cell culture is considered particularly advantageous due to the drawbacks associated with traditional methods which grow these viruses in eggs. For example, the growth of influenza viruses in eggs requires long lead times of up to six months which can be problematic because influenza vaccines need to be changed frequently because the influenza virus undergoes rapid mutation and vaccines often need to be available at short notice, especially during a pandemic.

Vaccine compositions, in particular influenza vaccines, are generally based either on live virus or on inactivated virus. Inactivated vaccines may be based on whole virions, ‘split’ virions, or on purified surface antigens. Antigens can also be presented in the form of virosomes. The invention can be used for any of these types of vaccine.

Where an inactivated virus is used, the vaccine may comprise whole virion, split virion, or purified surface antigens (including hemagglutinin and, usually, also including neuraminidase). Chemical means for inactivating a virus include treatment with an effective amount of one or more of the following agents: detergents, formaldehyde, β-propiolactone, methylene blue, psoralen, carboxyfullerene (C60), binary ethylamine, acetyl ethyleneimine, or combinations thereof. Non-chemical methods of viral inactivation are known in the art, such as for example UV light or gamma irradiation.

Virions can be harvested from virus-containing fluids, e.g. allantoic fluid or cell culture supernatant, by various methods. For example, a purification process may involve zonal centrifugation using a linear sucrose gradient solution that includes detergent to disrupt the virions. Antigens may then be purified, after optional dilution, by diafiltration.

Split virions are obtained by treating purified virions with detergents (e.g. ethyl ether, polysorbate 80, deoxycholate, tri-N-butyl phosphate, Triton X-100, Triton N101, cetyltrimethylammonium bromide, Tergitol NP9, etc.) to produce subvirion preparations, including the ‘Tween-ether’ splitting process. Methods of splitting influenza viruses, for example are well known in the art e.g. see refs. 14-19, etc. Splitting of the virus is typically carried out by disrupting or fragmenting whole virus, whether infectious or non-infectious with a disrupting concentration of a splitting agent. The disruption results in a full or partial solubilisation of the virus proteins, altering the integrity of the virus. Preferred splitting agents are non-ionic and ionic (e.g. cationic) surfactants e.g. alkylglycosides, alkylthioglycosides, acyl sugars, sulphobetaines, betains, polyoxyethylenealkylethers, N,N-dialkyl-Glucamides, Hecameg, alkylphenoxy-polyethoxyethanols, NP9, quaternary ammonium compounds, sarcosyl, CTABs (cetyl trimethyl ammonium bromides), tri-N-butyl phosphate, Cetavlon, myristyltrimethylammonium salts, lipofectin, lipofectamine, and DOT-MA, the octyl- or nonylphenoxy polyoxyethanols (e.g. the Triton surfactants, such as Triton X-100 or Triton N101), polyoxyethylene sorbitan esters (the Tween surfactants), polyoxyethylene ethers, polyoxyethlene esters, etc. One useful splitting procedure uses the consecutive effects of sodium deoxycholate and formaldehyde, and splitting can take place during initial virion purification (e.g. in a sucrose density gradient solution). Thus a splitting process can involve clarification of the virion-containing material (to remove non-virion material), concentration of the harvested virions (e.g. using an adsorption method, such as CaHPO₄ adsorption), separation of whole virions from non-virion material, splitting of virions using a splitting agent in a density gradient centrifugation step (e.g. using a sucrose gradient that contains a splitting agent such as sodium deoxycholate), and then filtration (e.g. ultrafiltration) to remove undesired materials. Split virions can usefully be resuspended in sodium phosphate-buffered isotonic sodium chloride solution. Examples of split influenza vaccines are the BEGRIVAC™, FLUARIX™, FLUZONE™ and FLUSHIELD™ products.

Another form of inactivated antigen is the virosome [20] (nucleic acid free viral-like liposomal particles). Virosomes can be prepared by solubilization of virus with a detergent followed by removal of the nucleocapsid and reconstitution of the membrane containing the viral glycoproteins. An alternative method for preparing virosomes involves adding viral membrane glycoproteins to excess amounts of phospholipids, to give liposomes with viral proteins in their membrane.

Purified influenza virus surface antigen vaccine compositions comprise the surface antigens hemagglutinin and, typically, also neuraminidase. Processes for preparing these proteins in purified form are well known in the art. The OPTAFLU™, CELTURA™, FLUVIRIN™, AGRIPPAL™ and INFLUVAC™ products are influenza subunit vaccines.

HA is the main immunogen in current inactivated influenza vaccine compositions, and vaccine doses are standardised by reference to HA levels, typically measured by SRiD. Existing vaccines typically contain about 15 μg of HA per strain, although lower doses can be used e.g. for children, or in pandemic situations, or when using an adjuvant. Fractional doses such as ½ (i.e. 7.5 μg HA per strain), ¼ and ⅛ have been used, as have higher doses (e.g. 3× or 9× doses [21,22]). Thus vaccines may include between 0.1 and 150 μg of HA per influenza strain per vaccine dose, preferably between 0.1 and 50 μg e.g. 0.1-20 μg, 0.1-15 μg, 0.1-10 μg, 0.1-7.5 μg, 0.5-5 μg, etc. Particular doses include e.g. about 45, about 30, about 15, about 10, about 7.5, about 5, about 3.8, about 3.75, about 1.9, about 1.5, etc. per strain per dose.

The invention may also be practised with compositions that contain live virus antigens, such as live influenza antigens. Such compositions are usually prepared by purifying virions from virion-containing fluids. For example, the fluids may be clarified by centrifugation, and stabilized with buffer (e.g. containing sucrose, potassium phosphate, and monosodium glutamate). Various forms of influenza virus vaccine are currently available (e.g. see chapters 17 & 18 of reference 23). Live virus vaccines include MedImmune's FLUMIST™ product (trivalent live virus vaccine).

In influenza vaccine compositions the influenza virus may be attenuated. The influenza virus may be temperature-sensitive. The influenza virus may be cold-adapted. These three features are particularly useful when using live virus as an antigen.

For live vaccines, dosing is measured by median tissue culture infectious dose (TCID₅₀) rather than HA content, and a TCID₅₀ of between 10⁶ and 10⁸ (preferably between 10^{6.5}-10^{7.5}) per strain is typical.

Influenza strains used with the invention may have a natural HA as found in a wild-type virus, or a modified HA. For instance, it is known to modify HA to remove determinants (e.g. hyper-basic regions around the HA1/HA2 cleavage site) that cause a virus to be highly pathogenic in avian species. The use of reverse genetics facilitates such modifications.

Influenza virus strains for use in vaccines change from season to season. In inter-pandemic periods, vaccines typically include two influenza A strains (H1N1 and H3N2) and one influenza B strain, and trivalent vaccines are typical. The invention may also use pandemic viral strains (i.e. strains to which the vaccine recipient and the general human population are immunologically naïve, in particular of influenza A virus), such as H2, H5, H7 or H9 subtype strains, and influenza vaccines for pandemic strains may be monovalent or may be based on a normal trivalent vaccine supplemented by a pandemic strain. Depending on the season and on the nature of the antigen included in the vaccine, however, the invention may protect against one or more of HA subtypes H1, H2, H3, H4, H5, H6, H7, H8, H9, H10, H11, H12, H13, H14, H15 or H16. The invention may protect against one or more of influenza A virus NA subtypes N1, N2, N3, N4, N5, N6, N7, N8 or N9.

As well as being suitable for immunizing against inter-pandemic strains, the compositions of the invention are particularly useful for immunizing against pandemic or potentially-pandemic strains. Thus, the compositions may comprise an antigen from a pandemic or a potentially-pandemic influenza strain. The characteristics of an influenza strain that give it the potential to cause a pandemic outbreak are: (a) it contains a new hemagglutinin compared to the hemagglutinins in currently-circulating human strains, i.e. one that has not been evident in the human population for over a decade (e.g. H2), or has not previously been seen at all in the human population (e.g. H5, H6 or H9, that have generally been found only in bird populations), such that the human population will be immunologically naïve to the strain's hemagglutinin; (b) it is capable of being transmitted horizontally in the human population; and (c) it is pathogenic to humans. A virus with H5 hemagglutinin type is preferred for immunizing against pandemic influenza, such as a H5N1 strain. Other possible strains include H5N3, H9N2, H2N2, H7N1 and H7N7, and any other emerging potentially pandemic strains. The invention is particularly suitable for protecting against potential pandemic virus strains that can or have spread from a non-human animal population to humans, for example a swine-origin H1N1 or H3N2 influenza strain. The compositions of the invention are then suitable for vaccinating humans as well as non-human animals.

Other strains whose antigens can usefully be included in the compositions are strains which are resistant to antiviral therapy (e.g. resistant to oseltamivir [24] and/or zanamivir), including resistant pandemic strains [25].

Compositions of the invention may include antigen(s) from one or more (e.g. 1, 2, 3, 4 or more) influenza virus strains, including influenza A virus and/or influenza B virus. Where a vaccine includes more than one strain of influenza, the different strains are typically grown separately and are mixed after the viruses have been harvested and antigens have been prepared. Thus a process of the invention may include the step of mixing antigens from more than one influenza strain. A trivalent vaccine is typical, including antigens from two influenza A virus strains and one influenza B virus strain. A tetravalent vaccine is also useful [26], including antigens from two influenza A virus strains and two influenza B virus strains, or three influenza A virus strains and one influenza B virus strain.

The compositions of the invention may comprise recombinant proteins. The recombinant proteins are preferably produced in mammalian cell culture. This is preferred because recombinant proteins are frequently used as therapeutics and growth in mammalian cells allows for proper protein folding, assembly and post-translational modification[27]. Thus, the quality and efficacy of a protein can be superior when expressed in mammalian cells versus other hosts such as bacteria, plants and yeast. Compositions that comprise recombinant proteins which are frequently produced in cell culture and whose productions process may therefore benefit from the present invention include anti-cancer drugs (such as Vectibix™, Campath™ or Rituxan™), growth hormones, insulin, tissue plasminogen activator (tPA), erythropoietin (Aranesp™) and blood factors (such as Factor VIII (ReFacto™) and Factor IX (Benefix™)).

Methods for producing recombinant proteins in cell culture are known in the art [28]. The recombinant proteins may be prepared in recombinant form by expression of their encoding nucleic acid molecules in vectors contained within the host cell. Generally, any system or vector that is suitable to maintain, propagate or express nucleic acid molecules to produce a recombinant protein may be used. The appropriate nucleotide sequence may be inserted into an expression system by any of a variety of well-known and routine techniques, such as, for example, those described in reference 28. Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

### Cells

The invention can be practised with any eukaryotic or prokaryotic cell that allows the production of the biological product of interest. The invention will typically use a cell line although, for example, primary cells may be used as an alternative. The cell will typically be mammalian. Suitable mammalian cells include, but are not limited to, hamster, cattle, primate (including humans and monkeys) and dog cells. Various cell types may be used, such as kidney cells, fibroblasts, retinal cells, lung cells, etc. Examples of suitable hamster cells are the cell lines having the names BHK21 or HKCC. Suitable monkey cells are e.g. African green monkey cells, such as kidney cells as in the Vero cell line[29-31]. Suitable dog cells are e.g. kidney cells, as in the CLDK and MDCK cell lines.

Further suitable cells include, but are not limited to: CHO; 293T; BHK; MRC 5; PER.C6 [32]; FRhL2; WI-38; etc. Suitable cells are widely available e.g. from the American Type Cell Culture (ATCC) collection[5], from the Coriell Cell Repositories [6], or from the European Collection of Cell Cultures (ECACC). For example, the ATCC supplies various different Vero cells under catalogue numbers CCL 81, CCL 81.2, CRL 1586 and CRL-1587, and it supplies MDCK cells under catalogue number CCL 34. PER.C6 is available from the ECACC under deposit number 96022940.

Preferred cells (particularly for growing influenza viruses) for use in the invention are MDCK cells [33-35], derived from Madin Darby canine kidney. The original MDCK cells are available from the ATCC as CCL 34. It is preferred that derivatives of these cells or other MDCK cells are used. Such derivatives were described, for instance, in reference 33 which discloses MDCK cells that were adapted for growth in suspension culture (‘MDCK 33016’ or ‘33016-PF’, deposited as DSM ACC 2219; see also ref 33). Furthermore, reference 36 discloses MDCK-derived cells that grow in suspension in serum free culture (′B-702′, deposited as FERM BP-7449). In some embodiments, the MDCK cell line used may be tumorigenic. It is also envisioned to use non-tumorigenic MDCK cells. For example, reference 37 discloses non tumorigenic MDCK cells, including ‘MDCK-S’ (ATCC PTA-6500), ‘MDCK-SF101’ (ATCC PTA-6501), ‘MDCK-SF102’ (ATCC PTA-6502) and ‘MDCK-SF103’ (ATCC PTA-6503). Reference 38 discloses MDCK cells with high susceptibility to infection, including ‘MDCK.5F1’ cells (ATCC CRL 12042).

It is possible to use a mixture of more than one cell type to practise the methods of the present invention. However, it is preferred that the methods of the invention are practised with a single cell type e.g. with monoclonal cells. Preferably, the cells used in the methods of the present invention are from a single cell line.

Preferably, the cells are cultured in the absence of serum, to avoid a common source of contaminants. Various serum-free media for eukaryotic cell culture are known to the person skilled in the art (e.g. Iscove's medium, ultra CHO medium (BioWhittaker), EX-CELL (JRH Biosciences)). Furthermore, protein-free media may be used (e.g. PF-CHO (JRH Biosciences)). Otherwise, the cells for replication can also be cultured in the customary serum-containing media (e.g. MEM or DMEM medium with 0.5% to 10% of fetal calf serum).

The cells may be in adherent culture or in suspension culture. Microcarrier cultures can be used. In some embodiments, the cells may be adapted for growth in suspension.

Multiplication of the cells can be conducted in accordance with methods known to those of skill in the art. For example, the cells can be cultivated in a perfusion system using ordinary support methods like centrifugation or filtration. Moreover, the cells can be multiplied according to the invention in a fed-batch system before infection. In the context of the present invention, a culture system is referred to as a fed-batch system in which the cells are initially cultured in a batch system and depletion of nutrients (or part of the nutrients) in the medium is compensated by controlled feeding of concentrated nutrients. It can be advantageous to adjust the pH value of the medium during multiplication of cells before infection to a value between pH 6.6 and pH 7.8 and especially between a value between pH 7.2 and pH 7.3. Culturing of cells preferably occurs at a temperature between 30 and 40° C. In step (iii), the cells are preferably cultured at a temperature of between 30° C. and 36° C. or between 32° C. and 34° C. or at 33° C. This is particularly preferred where the method of the invention is used to produce influenza virus, as it has been shown that incubation of infected cells in this temperature range results in production of a virus that results in improved efficacy when formulated into a vaccine[39].

The oxygen partial pressure can be adjusted during culturing before infection preferably at a value between 25% and 95% and especially at a value between 35% and 60%. The values for the oxygen partial pressure stated in the context of the invention are based on saturation of air. Infection of cells occurs at a cell density of preferably about 8-25×10⁵ cells/mL in the batch system or preferably about 5-20×10⁶ cells/mL in the perfusion system. The cells can be infected with a viral dose (MOI value, “multiplicity of infection”; corresponds to the number of virus units per cell at the time of infection) between 10⁻⁸ and 10, preferably between 0.0001 and 0.5.

The methods according to the invention can also include harvesting and isolation of recombinant proteins or viruses or the proteins generated by the viruses. During isolation of proteins or viruses, the cells are separated from the culture medium by standard methods like separation, filtration or ultrafiltration. The proteins or the viruses are then concentrated according to methods sufficiently known to those skilled in the art, like gradient centrifugation, filtration, precipitation, chromatography, etc., and then purified. It is also preferred according to the invention that the viruses are inactivated during or after purification. Virus inactivation can occur, for example, by β-propiolactone or formaldehyde at any point within the purification process.

Systems for the expression of recombinant proteins are known in the art (see for example reference 28). Recombinant proteins will usually be expressed cells using expression systems which comprise a promoter (such as SV40 early promoter, cytomegalovirus (CMV) promoter, mouse mammary tumor virus LTR promoter, adenovirus major late promoter (Ad MLP), or herpes simplex virus promoter), a polyadenylation signal and a transcription termination sequence. Enhancers, introns with functional splice donor and acceptor sites, and leader sequences may also be included in an expression construct. The recombinant proteins may be expressed intracellularly in mammalian cells. Alternatively, the recombinant protein can also be secreted from the cell into the growth medium by creating chimeric DNA molecules that encode a fusion protein comprised of a leader sequence fragment that provides for secretion of the foreign protein in mammalian cells.

### Repetitive Elements

The invention can, in principle, be practised with any repetitive element which is present in the genome of a cell. It is preferred, however, to practise the invention with repetitive elements which are present with more than one copy number in the genome of the host cell (for example with more than 10, more than 100, more than 500 or more than 1000 copy numbers). This is preferred because genome segments which are present in high copy numbers are more easily detectable in amplification reactions and so the sensitivity of the methods of the invention can be improved.

Preferably the fragment of the repetitive element and/or the housekeeping gene which is amplified has a length which is equivalent to the length of known oncogenes in the host cell. Oncogenes pose the biggest problem to the safety of the vaccine when they are present as full-length genes but DNA removal steps used during vaccine manufacture often fragment the DNA. By amplifying a fragment of a repetitive element with the approximate same length of a known oncogene the methods of the invention also allow the skilled person to assess the likelihood that a full-length oncogene is present. For example, if no fragments of the repetitive element can be amplified it is likely that no host cell DNA is present in the cell which is long enough to encode a full-length oncogene. The DNA fragment which is amplified can have a length of 700-1300 bp, 800-1200 bp, 900-1100 bp or about 1000 bp.

Genome segments which are present in high copy numbers and which are therefore particularly suitable for use in the invention include retrotransposons, such as long terminal repeat (LTR) retrotransposons and non-LTR retrotransposons.

It is particularly preferred to use long interspersed elements (LINEs), short interspersed elements (SINEs), SVAs or pseudogenes in the invention because these are amongst the most abundant genomic elements and are found in a wide variety of species.

LINEs are approximately 6 kb long and occur with a frequency of about 10,000-100,000 copies per haploid mammalian genome (5% to 17% of genome). In particular, LINE1 (L1) is the most abundant self-replicating transposon in the genome and has approximately 80,000 copies in the human genome. Close to 10,000 of these LINE elements are full length and contain two long open reading frames (ORF1 and ORF2), both of which encode proteins that are required for retrotransposition. The sequence of the ORF2 sequence of a LINE1 element is shown in SEQ ID NO: 3. LINES are particularly preferred for use with the invention because they are present in high copy numbers. Furthermore, they often have a length of more than 1000 bp and so it is possible to amplify fragments which have the approximate size of a known oncogene.

SINE elements are distinguished from LINE elements based on their length and are by definition less than 500 bp in length. The most abundant SINEs are the Alu elements [40]. Alu elements have a length of about 300 bp and are estimated to be present with more than 500,000 copy numbers. Structurally, Alu elements have a two-part structure with a 5′ region containing an RNA polymerase III promoter and a 3′ region which is slightly longer than the 5′ region. The 5′ region and the 3′ region are separated by an intervening, central A-rich region that consists of the sequence 5′-A₅TACA₆-3′ (SEQ ID NO: 4). A typical Alu element ends with a poly(A) tail.

SVA elements [41] are believed to have originated from a combination of SINE-R, VNTR, and Alu elements. SVAs include a 490 bp part of SINE-R sequences derived from the 3′ end of the env gene, part of the 3′ long terminal repeat of the human endogenous retrovirus K-10 (HERV-K10), a region containing a variable number of tandem repeats (VNTR) each consisting of 35-50 nucleotides, and Alu-like sequences. SVA elements are present in humans in about 2700 copies.

Pseudogenes are non-functional genes which have homology to a functional gene in the genome. There are approximately 20,000 pseudogenes in the human genome. For example, the ribosomal protein pseudogenes comprise a large family of pseudogenes with approximately 2000 copies in the genome.

LTR retrotransposons are amongst the most abundant genomic elements and can be present in the genome with up to a few million copies per haploid genome. Approximately 8% of the human genome is composed of such LTR transposons. LTR retrotransposons can broadly be divided into the copia/Ty1 and the gypsy/Ty3 families. LTR retrotransposons range in length from 100 bp to 5 kb in size the full-length elements are generally characterized by the presence of long terminal repeats (LTRs) at the 5′ and 3′ end.

### Housekeeping Genes

The invention can also be practised with any housekeeping gene which is present in the genome of a cell. It is preferred to practise the invention with housekeeping genes which are present with more than one copy number in the genome of the host cell (for example with more than 2, more than 5 or more than 10 copy numbers). This is preferred because genome segments which are present in high copy numbers are more easily detectable in amplification reactions and so the sensitivity of the methods of the invention can be improved.

Housekeeping genes are genes which are constitutively expressed in the cell and which usually encode genes that are important for the function of the cell. Examples of suitable housekeeping genes which can be used include GAPDH, β-actin, tubulin, hypoxanthine guanine phosphoribosyltransferase (HPRT), porphobilinogen deaminase (PBGD) and ribosomal proteins (such as RPL7 and RPL32).

### Determining the Copy Number of the Repetitive Element or Housekeeping Gene

The copy number of the repetitive element or the housekeeping gene can be determined by amplifying at least a fragment of the repetitive element or the housekeeping gene, diluting the amplified product in a dilution series and detecting the amplified product, for example by separation on an agarose gel. The copy number of the repetitive element or the housekeeping gene (Nᵣₑₚ) can be determined by the following formula in which ‘DL’ is the detection limit of the specific repetitive element or housekeeping gene under the given conditions (repetitive element/mL), ‘PCR(−)’ indicates the dilution factor of the first negative amplification signal and ‘dilution’ refers to the dilution factor of any initial dilution which may optionally have taken place after DNA extraction:

Nᵣₑₚ/mL=DL×PCR(−)×dilution

It was already explained above how the detection limit of the amplification reaction can be determined.

Nucleic acid amplification techniques (NAATs) which can be used for DNA amplification include thermal cycling techniques as well as isothermal techniques e.g. the polymerase chain reaction (PCR), the ligase chain reaction (LCR), rolling-circle amplification (RCA) [42], boomerang DNA amplification (BDA) [43], the Qb replicase system, the repair chain reaction (RCR), self-sustaining sequence replication (3SR), the strand displacement assay (SDA), etc.

These amplification techniques generally involve the use of one or more pairs of primers which hybridise to opposite strands of a double-stranded target. These primers need to be designed such that they amplify at least a fragment of the repetitive element or the housekeeping gene which is used. Methods in which the full-length repetitive element or housekeeping gene are amplified can also be used. Ways of providing suitable primers are known to the skilled person.

The amplified product can be detected by any method which allows the detection of DNA. For example, the product can be detected by separation on an agarose gel. It may also be detected by assays, such as the Threshold™ system or Absorbance DNA Quantitation. It may also be detected by other methods, such as Molecular Counting.

### Pharmaceutical Compositions

Compositions of the invention are pharmaceutically acceptable. They usually include components in addition to the antigens e.g. they typically include one or more pharmaceutical carrier(s) and/or excipient(s). As described below, adjuvants may also be included. A thorough discussion of such components is available in reference 44.

Compositions, in particular vaccine compositions, will generally be in aqueous form. However, some vaccines may be in dry form, e.g. in the form of injectable solids or dried or polymerized preparations on a patch.

Vaccine compositions may include preservatives such as thiomersal or 2-phenoxyethanol. It is preferred, however, that the vaccine should be substantially free from (i.e. less than 5 μg/ml) mercurial material e.g. thiomersal-free [45]. Vaccines containing no mercury are more preferred. α-tocopherol succinate can be included as an alternative to mercurial compounds. Preservative-free vaccines are particularly preferred.

To control tonicity, it is preferred to include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride, calcium chloride, etc.

Vaccine compositions will generally have an osmolality of between 200 mOsm/kg and 400 mOsm/kg, preferably between 240-360 mOsm/kg, and will more preferably fall within the range of 290-310 mOsm/kg. Osmolality has previously been reported not to have an impact on pain caused by vaccination [46], but keeping osmolality in this range is nevertheless preferred.

Vaccine compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a vaccine composition will generally be between 5.0 and 8.1, and more typically between 6.0 and 8.0 e.g. 6.5 and 7.5, or between 7.0 and 7.8. A process of the invention may therefore include a step of adjusting the pH of the bulk vaccine prior to packaging.

The vaccine composition is preferably sterile. The vaccine composition is preferably non-pyrogenic e.g. containing <1 EU (endotoxin unit, a standard measure) per dose, and preferably <0.1 EU per dose. The vaccine composition is preferably gluten-free.

Vaccine compositions of the invention may include detergent e.g. a polyoxyethylene sorbitan ester surfactant (known as ‘Tweens’), an octoxynol (such as octoxynol-9 (Triton X-100) or t-octylphenoxypolyethoxyethanol), a cetyl trimethyl ammonium bromide (‘CTAB’), or sodium deoxycholate, particularly for a split or surface antigen vaccine. The detergent may be present only at trace amounts. Thus the vaccine may include less than 1 mg/ml of each of octoxynol-10 and polysorbate 80. Other residual components in trace amounts could be antibiotics (e.g. neomycin, kanamycin, polymyxin B).

A vaccine composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

The composition may have a volume of 0.1-1 mL, for example 0.2-0.8 mL, 0.3-0.7 mL, 0.4 to 0.6 mL or about 0.5 mL. Influenza vaccines are typically administered in a dosage volume of about 0.5 ml, although a half dose (i.e. about 0.25 ml) may be administered to children.

Compositions and kits are preferably stored at between 2° C. and 8° C. They should not be frozen. They should ideally be kept out of direct light.

### Host Cell DNA

Where a biological product, such as a virus, has been produced isolated and/or grown on a cell line, it is standard practice to minimize the amount of residual cell line DNA in the final composition, in order to minimize any oncogenic activity of the DNA.

Thus a composition (in particular a vaccine composition) according to the invention preferably contains less than 10 ng (preferably less than ing, and more preferably less than 100 pg) of residual host cell DNA per dose, although trace amounts of host cell DNA may be present.

It is preferred that the average length of any residual host cell DNA is less than 500 bp e.g. less than 400 bp, less than 300 bp, less than 200 bp, less than 100 bp, etc.

Contaminating DNA can be removed during vaccine preparation using standard purification procedures e.g. chromatography, etc. Removal of residual host cell DNA can be enhanced by nuclease treatment e.g. by using a DNase. A convenient method for reducing host cell DNA contamination is disclosed in references 47 & 48, involving a two-step treatment, first using a DNase (e.g. Benzonase), which may be used during viral growth, and then a cationic detergent (e.g. CTAB), which may be used during virion disruption. Treatment with an alkylating agent, such as β-propiolactone, can also be used to remove host cell DNA, and advantageously may also be used to inactivate virions [49].

The amount of residual host cell DNA can be measured. Thus, in a further aspect, the invention provides methods of using a repetitive element (in particular LINE elements, SVA elements or pseudogenes) or a housekeeping gene to determine the amount of residual host cell DNA in a composition comprising a biological product produced in a host cell. These methods may comprise the steps of (a) amplifying at least a fragment of a repetitive element or of a housekeeping gene of the host cell; (b) using the amplified DNA to determine the copy number of the repetitive element or the housekeeping gene; and (c) using the copy number of the repetitive element or the housekeeping gene to calculate the amount of residual host cell DNA in the composition.

In order to calculate the amount of residual host cell DNA in a composition it is necessary to know the correlation between the copy number of the repetitive element or the housekeeping gene and the total amount of DNA. This can be determined by amplifying the repetitive element or the housekeeping from the total DNA in a cell, determining the copy number of the repetitive element or the housekeeping gene and calculating the ratio between the copy number of the repetitive element or the housekeeping gene and the total amount of DNA in the cell.

### Adjuvants

Compositions of the invention may advantageously include an adjuvant, which can function to enhance the immune responses (humoral and/or cellular) elicited in a subject who receives the composition. Preferred adjuvants comprise oil-in-water emulsions. Various such adjuvants are known, and they typically include at least one oil and at least one surfactant, with the oil(s) and surfactant(s) being biodegradable (metabolisable) and biocompatible. The oil droplets in the emulsion are generally less than 5 μm in diameter, and ideally have a sub-micron diameter, with these small sizes being achieved with a microfluidiser to provide stable emulsions. Droplets with a size less than 220 nm are preferred as they can be subjected to filter sterilization.

The emulsion can comprise oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein. Squalane, the saturated analog to squalene, is also a preferred oil. Fish oils, including squalene and squalane, are readily available from commercial sources or may be obtained by methods known in the art. Another preferred oil is α-tocopherol (see below).

### Mixtures of Oils can be Used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are Tween 80 (polyoxyethylene sorbitan monooleate), Span 85 (sorbitan trioleate), lecithin and Triton X-100.

Mixtures of surfactants can be used e.g. Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Where the vaccine contains a split virus, it is preferred that it contains free surfactant in the aqueous phase. This is advantageous as the free surfactant can exert a ‘splitting effect’ on the antigen, thereby disrupting any unsplit virions and/or virion aggregates that might otherwise be present. This can improve the safety of split virus vaccines [50].

Preferred emulsions have an average droplets size of <1 μm e.g. ≦750 nm, ≦500 nm, ≦400 nm, ≦300 nm, ≦250 nm, ≦220 nm, ≦200 nm, or smaller. These droplet sizes can conveniently be achieved by techniques such as microfluidisation.

Specific oil-in-water emulsion adjuvants useful with the invention include, but are not limited to:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [51-53], as described in more detail in Chapter 10 of ref 54 and chapter 12 of ref 55. The MF59 emulsion advantageously includes citrate ions e.g. 10 mM sodium citrate buffer.
An emulsion comprising squalene, a tocopherol, and polysorbate 80. The emulsion may include phosphate buffered saline. These emulsions may have by volume from 2 to 10% squalene, from 2 to 10% tocopherol and from 0.3 to 3% polysorbate 80, and the weight ratio of squalene:tocopherol is preferably <1 (e.g. 0.90) as this can provide a more stable emulsion. Squalene and polysorbate 80 may be present at a volume ratio of about 5:2 or at a weight ratio of about 11:5. Thus the three components (squalene, tocopherol, polysorbate 80) may be present at a weight ratio of 1068:1186:485 or around 55:61:25. One such emulsion (‘AS03’) can be made by dissolving Tween 80 in PBS to give a 2% solution, then mixing 90 ml of this solution with a mixture of (5 g of DL a tocopherol and 5 ml squalene), then microfluidising the mixture. The resulting emulsion may have submicron oil droplets e.g. with an average diameter of between 100 and 250 nm, preferably about 180 nm. The emulsion may also include a 3-de-O-acylated monophosphoryl lipid A (3d MPL). Another useful emulsion of this type may comprise, per human dose, 0.5-10 mg squalene, 0.5-11 mg tocopherol, and 0.1-4 mg polysorbate 80 e.g. in the ratios discussed above.
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 μg/ml polysorbate 80, 110 μg/ml Triton X-100 and 100 μg/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [56] (0.05-1% Thr-MDP, 5% squalane, 2.5% Pluronic L121 and 0.2% polysorbate 80). It can also be used without the Thr-MDP, as in the “AF” adjuvant [57] (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm [58]. The emulsion may also include one or more of: alditol; a cryoprotective agent (e.g. a sugar, such as dodecylmaltoside and/or sucrose); and/or an alkylpolyglycoside. The emulsion may include a TLR4 agonist [59]. Such emulsions may be lyophilized.
An emulsion of squalene, poloxamer 105 and Abil-Care [60]. The final concentration (weight) of these components in adjuvanted vaccines are 5% squalene, 4% poloxamer 105 (pluronic polyol) and 2% Abil-Care 85 (Bis-PEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in reference 61, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 62, produced by addition of aliphatic amine to desacylsaponin via the carboxyl group of glucuronic acid), dimethyidioctadecylammonium bromide and/or N,N-dioctadecyl-N,N-bis(2-hydroxyethyl)propanediamine
An emulsion in which a saponin (e.g. QuilA or QS21) and a sterol (e.g. a cholesterol) are associated as helical micelles [63].
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) [64].
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) [64].

In some embodiments an emulsion may be mixed with antigen extemporaneously, at the time of delivery, and thus the adjuvant and antigen may be kept separately in a packaged or distributed vaccine, ready for final formulation at the time of use. In other embodiments an emulsion is mixed with antigen during manufacture, and thus the composition is packaged in a liquid adjuvanted form. The antigen will generally be in an aqueous form, such that the vaccine is finally prepared by mixing two liquids. The volume ratio of the two liquids for mixing can vary (e.g. between 5:1 and 1:5) but is generally about 1:1. Where concentrations of components are given in the above descriptions of specific emulsions, these concentrations are typically for an undiluted composition, and the concentration after mixing with an antigen solution will thus decrease.

### Packaging of Vaccine Compositions

Suitable containers for compositions of the invention (or kit components) include vials, syringes (e.g. disposable syringes), nasal sprays, etc. These containers should be sterile.

Where a composition/component is located in a vial, the vial is preferably made of a glass or plastic material. The vial is preferably sterilized before the composition is added to it. To avoid problems with latex-sensitive patients, vials are preferably sealed with a latex-free stopper, and the absence of latex in all packaging material is preferred. The vial may include a single dose of vaccine, or it may include more than one dose (a ‘multidose’ vial) e.g. 10 doses. Preferred vials are made of colourless glass.

A vial can have a cap (e.g. a Luer lock) adapted such that a pre-filled syringe can be inserted into the cap, the contents of the syringe can be expelled into the vial (e.g. to reconstitute lyophilised material therein), and the contents of the vial can be removed back into the syringe. After removal of the syringe from the vial, a needle can then be attached and the composition can be administered to a patient. The cap is preferably located inside a seal or cover, such that the seal or cover has to be removed before the cap can be accessed. A vial may have a cap that permits aseptic removal of its contents, particularly for multidose vials.

Where a component is packaged into a syringe, the syringe may have a needle attached to it. If a needle is not attached, a separate needle may be supplied with the syringe for assembly and use. Such a needle may be sheathed. Safety needles are preferred. 1-inch 23-gauge, 1-inch 25-gauge and ⅝-inch 25-gauge needles are typical. Syringes may be provided with peel-off labels on which the lot number, influenza season and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of vaccine. The syringe will generally have a tip cap to seal the tip prior to attachment of a needle, and the tip cap is preferably made of a butyl rubber. If the syringe and needle are packaged separately then the needle is preferably fitted with a butyl rubber shield. Preferred syringes are those marketed under the trade name “Tip-Lok”™.

Containers may be marked to show a half-dose volume e.g. to facilitate delivery to children. For instance, a syringe containing a 0.5 ml dose may have a mark showing a 0.25 ml volume.

Where a glass container (e.g. a syringe or a vial) is used, then it is preferred to use a container made from a borosilicate glass rather than from a soda lime glass.

A kit or composition may be packaged (e.g. in the same box) with a leaflet including details of the vaccine e.g. instructions for administration, details of the antigens within the vaccine, etc. The instructions may also contain warnings e.g. to keep a solution of adrenaline readily available in case of anaphylactic reaction following vaccination, etc.

### Methods of Treatment, and Administration of the Vaccine

The invention provides a vaccine manufactured according to the invention. These vaccine compositions are suitable for administration to human or non-human animal subjects, such as pigs, and the invention provides a method of raising an immune response in a subject, comprising the step of administering a composition of the invention to the subject. The invention also provides a composition of the invention for use as a medicament, and provides the use of a composition of the invention for the manufacture of a medicament for raising an immune response in a subject.

The immune response raised by these methods and uses will generally include an antibody response, preferably a protective antibody response. Methods for assessing antibody responses, neutralising capability and protection after influenza virus vaccination are well known in the art. Human studies have shown that antibody titers against hemagglutinin of human influenza virus are correlated with protection (a serum sample hemagglutination-inhibition titer of about 30-40 gives around 50% protection from infection by a homologous virus) [65]. Antibody responses are typically measured by hemagglutination inhibition, by microneutralisation, by single radial immunodiffusion (SRID), and/or by single radial hemolysis (SRH). These assay techniques are well known in the art.

Compositions of the invention can be administered in various ways. The most preferred immunisation route is by intramuscular injection (e.g. into the arm or leg), but other available routes include subcutaneous injection, intranasal[66-68], oral[69], intradermal[70, 71], transcutaneous, transdermal [72], etc.

Vaccines prepared according to the invention may be used to treat both children and adults. Influenza vaccines are currently recommended for use in pediatric and adult immunisation, from the age of 6 months. Thus a human subject may be less than 1 year old, 1-5 years old, 5-15 years old, 15-55 years old, or at least 55 years old. Preferred subjects for receiving the vaccines are the elderly (e.g. ≧50 years old, ≧60 years old, and preferably ≧65 years), the young (e.g. ≦5 years old), hospitalised subjects, healthcare workers, armed service and military personnel, pregnant women, the chronically ill, immunodeficient subjects, subjects who have taken an antiviral compound (e.g. an oseltamivir or zanamivir compound; see below) in the 7 days prior to receiving the vaccine, people with egg allergies and people travelling abroad. The vaccines are not suitable solely for these groups, however, and may be used more generally in a population. For pandemic strains, administration to all age groups is preferred.

Preferred compositions of the invention satisfy 1, 2 or 3 of the CPMP criteria for efficacy. In adults (18-60 years), these criteria are: (1) ≧70% seroprotection; (2) ≧40% seroconversion; and/or (3) a GMT increase of ≧2.5-fold. In elderly (>60 years), these criteria are: (1) ≧60% seroprotection; (2) ≧30% seroconversion; and/or (3) a GMT increase of ≧2-fold. These criteria are based on open label studies with at least 50 patients.

Treatment can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Administration of more than one dose (typically two doses) is particularly useful in immunologically naïve patients e.g. for people who have never received an influenza vaccine before, or for vaccinating against a new HA subtype (as in a pandemic outbreak).

Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Vaccines produced by the invention may be administered to patients at substantially the same time as (e.g. during the same medical consultation or visit to a healthcare professional or vaccination centre) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, a pneumococcal conjugate vaccine, etc. Administration at substantially the same time as a pneumococcal vaccine and/or a meningococcal vaccine is particularly useful in elderly patients.

Similarly, vaccines of the invention may be administered to patients at substantially the same time as (e.g. during the same medical consultation or visit to a healthcare professional) an antiviral compound, and in particular an antiviral compound active against influenza virus (e.g. oseltamivir and/or zanamivir). These antivirals include neuraminidase inhibitors, such as a (3R,4R,5S)-4-acetylamino-5-amino-3(1-ethylpropoxy)-1-cyclohexene-1-carboxylic acid or 5-(acetylamino)-4-[(aminoiminomethyl)-amino]-2,6-anhydro-3,4,5-trideoxy-D-glycero-D-galactonon-2-enonic acid, including esters thereof (e.g. the ethyl esters) and salts thereof (e.g. the phosphate salts). A preferred antiviral is (3R,4R,5S)-4-acetylamino-5-amino-3(1-ethylpropoxy)-1-cyclohexene-1-carboxylic acid, ethyl ester, phosphate (1:1), also known as oseltamivir phosphate (TAMIFLU™).

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

The different process steps may be performed at substantially the same time, or may be performed separately. They can be performed in the same location or in different locations, even in different countries e.g. the vaccine may be prepared in a place which is different from the place where the DNA safety factor is determined and/or it is prepared by a person which is different from the person who determines the safety factor.

Where animal (and particularly bovine) materials are used in the culture of cells, they should be obtained from sources that are free from transmissible spongiform encephalopathies (TSEs), and in particular free from bovine spongiform encephalopathy (BSE). Overall, it is preferred to culture cells in the total absence of animal-derived materials.

### BRIEF DESCRIPTION OF SEQUENCE LISTING

SEQ ID NO: 1 is a forward primer used to amplify a fragment of LINE ORF2
SEQ ID NO: 2 is a reverse primer used to amplify a fragment of LINE ORF2
SEQ ID NO: 3 is a 2078 bp fragment of LINE-ORF2
SEQ ID NO: 4 is the central A-rich region of a SINE element

### MODES FOR CARRYING OUT THE INVENTION

### Amplification of LINE Fragments

For the PCR assay to determine the LINE fragments in the samples, the primer sequences ACTGTAGTGAGAGATGAAGAGG (SEQ ID NO: 1) and GGCGTATACCTGTTCATAAT (SEQ ID NO: 2) are used which amplify a fragment of 1002 bp of ORF2 (SEQ ID NO. 3).

The fragment size of 1000 bp is half of the mean size of a human oncogene, which is reported with 1925 bp [73]. A long range polymerase from the company New England Biolabs is used as polymerase. The LINE fragment is amplified using an initial denaturing step for 2 min. at 94° C., followed by 35 cycles of DNA amplification (94° C. for 30 sec; 53° C. for 30 sec; 72° C. for 1 min) and a final elongation step at 72° C. for 4 min.

A dilution series of the PCR product is produced and the PCR product is analysed on an agarose gel. The evaluation of the results is performed by an end point evaluation. The first negative PCR signal in the dilution series is taken for the calculation. The detection limit (DL) of the 1002 bp fragment in the monobulk matrix and the ratio of LINE fragments to oncogenes in the MDCK genome (R) is incorporated into the subsequent calculation of the oncogenes in the sample.

Nine monobulks are measured in a two-fold determination with the PCR method. The first negative PCR signal (PCR(−)) in a dilution series is taken for the calculation. With this dilution factor, the detection limit of the 1002 bp fragment in the monobulk matrix and the ratio of the oncogenes per LINE the concentration of oncogenes in the monobulks can be calculated as follows:

c_{onco}=PCR(−)×DL×R

in which ‘DL’ is the detection limit of the specific repetitive element or housekeeping gene under the given conditions (repetitive element/mL) and ‘PCR(−)’ indicates the dilution factor of the first negative PCR signal. The results are as follows:

| | | | c_{onco} |
|---|---|---|---|
| Strain | Sample | PCR(−) | [oncogenes/mL] |
| A (H1N1) | A | 10¹ | 0.62 |
| | B | 10² | 6.2 |
| | C | 10² | 6.2 |
| H3(N2) | D | 10³ | 62 |
| | E | 10² | 6.2 |
| | F | 10² | 6.2 |
| B | G | 10¹ | 0.62 |
| | H | 10¹ | 0.62 |
| | I | 10² | 6.2 |

### Determination of the DNA Safety Factor

### DNA Safety Factor Scaled Up to 10 ng DNA

The DNA safety factor is calculated with relation to maximum allowed DNA content in a vaccine dose using the formula:

$$SF = \frac{{N}_{critical}^{oncogenes}}{{c}_{onco} \times \frac{10 ng}{{c}_{DNA}}}$$

With
   N_{critical}: Required number of oncogenes per dose to produce a tumor [Oncogenes]
   c_{onco}: Concentration of oncogenes in monobulk [Oncogenes/mL],
   c_{DNA}: DNA concentration in monobulk according to threshold [ng/mL].

The required number of oncogenes to produce a tumor is 1.6×10⁹ and the concentration of oncogenes in the monobulk is given by the PCR according to Table 1. Thus an example calculation for the H1N1 strain is:

$$SF = \frac{{N}_{critical}^{oncogenes}}{{c}_{onco} \times \frac{10 ng}{{c}_{DNA}}} = \frac{1.6 \cdot {10}^{9} oncogenes}{0.62 \frac{oncogenes}{mL} \times \frac{10 ng}{1.00 \frac{ng}{mL}}} = 3 \cdot {10}^{8}$$

The safety factor for the monobulk vaccines is shown in Table 2:

| | | c_{onco} | | DNA safety |
|---|---|---|---|---|
| Strain | Sample | [oncogenes/mL] | c_{DNA} [ng/mL] | factor |
| A (H1N1) | A | 0.62 | 1.00 | 3 × 10⁸ |
| | B | 6.2 | 1.17 | 3 × 10⁷ |
| | C | 6.2 | 0.90 | 2 × 10⁷ |
| H3(N2) | D | 62 | 8.43 | 2 × 10⁷ |
| | E | 6.2 | 17.27 | 4 × 10⁸ |
| | F | 6.2 | 11.70 | 3 × 10⁸ |
| B | G | 0.62 | 3.97 | 1 × 10⁹ |
| | H | 0.62 | 3.40 | 9 × 10⁸ |
| | I | 6.2 | 5.17 | 1 × 10⁸ |

### DNA Safety Factor Scaled Up to 10 ng DNA

A second consideration of the DNA safety factor is related to the volume of one dose. In the case of influenza vaccines one dose is usually equivalent to 0.5 mL.

The DNA safety factor is calculated by

$$SF = \frac{{N}_{critical}^{oncogenes}}{{c}_{onco} \times 0.5 mL}$$

With
   N_{critical}: Required number of oncogenes per dose to produce a tumour [Oncogenes]
   c_{onco}: Concentration of oncogenes in monobulk [Oncogenes/mL].

The required number of oncogenes to produce a tumour is 1.6×10⁹ and the concentration of oncogenes in the monobulk is given by the PCR. Thus an example calculation for the H1N1 strain is:

$$SF = \frac{{N}_{critical}^{oncogenes}}{{c}_{onco} \times 0.5 mL} = \frac{1.6 \cdot {10}^{9} oncogenes}{0.62 \frac{oncogenes}{mL} \times 0.5 mL} = 5 \cdot {10}^{9}$$

The safety factor for the monobulk vaccines is shown in Table 3:

| | | c_{onco} | |
|---|---|---|---|
| Strain | Sample | [oncogenes/mL] | DNA safety factor |
| A (H1N1) | A | 0.62 | 5 × 10⁹ |
| | B | 6.2 | 5 × 10⁸ |
| | C | 6.2 | 5 × 10⁸ |
| H3(N2) | D | 62 | 5 × 10⁷ |
| | E | 6.2 | 5 × 10⁸ |
| | F | 6.2 | 5 × 10⁸ |
| B | G | 0.62 | 5 × 10⁹ |
| | H | 0.62 | 5 × 10⁹ |
| | I | 6.2 | 5 × 10⁸ |

### DNA Safety Factor Scaled Up to the Active Ingredients (HA) Per Dose

This consideration of the DNA safety factor is related to the HA content of the monobulk. One final vaccine dose contains a total amount of 50 μg HA.

The DNA safety factor is calculated by

$$SF = \frac{{N}_{critical}^{oncogenes}}{{c}_{onco} \times \frac{50 µg}{{c}_{HA}}}$$

With
   N_{critical}: Required number of oncogenes per dose to produce a tumour [Oncogenes]
   c_{onco}: Concentration of oncogenes in monobulk [Oncogenes/mL]
   c_{HA}: HA concentration in the monobulk (μg/mL)

The required number of oncogenes to produce a tumour is 1.6×10⁹ and the concentration of oncogenes in the monobulk is given by the PCR. The amount of HA antigen is determined by SRID to be 245 μg/mL.

Thus an example calculation for the H1N1 strain is:

$$SF = \frac{{N}_{critical}^{oncogenes}}{{c}_{onco} \times \frac{50 \frac{µg}{dose}}{{c}_{HA}}} = \frac{1.6 \cdot {10}^{9} oncogenes}{0.62 \frac{oncogenes}{mL} \times \frac{50 \frac{µg}{dose}}{245 \frac{µg}{mL}}} = 1 \cdot {10}^{10}$$

The safety factor for the monobulk vaccines is shown in Table 4

| | | c_{onco} | | DNA safety |
|---|---|---|---|---|
| Strain | Sample | [oncogenes/mL] | cHA [μg/mL] | factor |
| A (H1N1) | A | 0.62 | 245 | 1 × 10¹⁰ |
| | B | 6.2 | 297 | 2 × 10⁹ |
| | C | 6.2 | 330 | 2 × 10⁹ |
| H3(N2) | D | 62 | 410 | 2 × 10⁸ |
| | E | 6.2 | 624 | 3 × 10⁹ |
| | F | 6.2 | 470 | 2 × 10⁹ |
| B | G | 0.62 | 434 | 2 × 10¹⁰ |
| | H | 0.62 | 430 | 2 × 10¹⁰ |
| | I | 6.2 | 451 | 2 × 10⁹ |

It will be understood that the invention has been described by way of example only and modifications may be made whilst remaining within the scope and spirit of the invention.

### REFERENCES

[1] Sheng-Fowler et al. (2010) Int J Biol Sci; 6(2):151-162
[2] Yang et al. (2010) Vaccine. 28(19):3308-11
[3] Sheng-Fowler L, et al. (2009). Biologicals 37:259-269
[4] Gregory, T. R. (2012). Animal Genome Size Database. http://www.genomesize.com
[5] http://www.atcc.org/
[6] http://locus.umdnj.edu/
[7] Briggs (1991) J Parenter Sci Technol. 45:7-12.
[8] Ji et al. (2002) Biotechniques. 32:1162-7.
[9] Lahijani et al. (1998) Hum Gene Ther. 9:1173-80.
[10] Lokteff et al. (2001) Biologicals. 29:123-32.
[11] Williams (1993) Vet Microbiol 37:253-262.
[12] Fitzgerald & Needy (1986) Dev Biol Stand 64:73-79.
[13] Bradford, M. M. (1976) Anal. Biochem. 72: 248-254
[14] WO02/28422
[15] WO02/067983
[16] WO02/074336
[17] WO01/21151
[18] WO02/097072
[19] WO2005/113756
[20] Huckriede et al. (2003) Methods Enzymol 373:74-91
[21] Treanor et al. (1996) J Infect Dis 173:1467-70
[22] Keitel et al. (1996) Clin Diagn Lab Immunol 3:507-10
[23] Vaccines. (eds. Plotkins & Orenstein). 4th edition, 2004, ISBN: 0-7216-9688-0
[24] Herlocher et al. (2004) J Infect Dis 190(9):1627-30
[25] Le et al. (2005) Nature 437(7062):1108
[26] WO2008/068631
[27] Wurm F. M. (2004) Nat Biotechnol. 22(11):1393-8.
[28] Sambrook (1989) Molecular Cloning; A Laboratory Manual, Second Edition
[29] Kistner et al. (1998) Vaccine 16:960-8
[30] Kistner et al. (1999) Dev Biol Stand 98:101-110
[31] Bruhl et al. (2000) Vaccine 19:1149-58
[32] Pau et al. (2001) Vaccine 19:2716-21
[33] WO97/37000
[34] Brands et al. (1999) Dev Biol Stand 98:93-100
[35] Halperin et al. (2002) Vaccine 20:1240-7
[36] EP-A-1260581 (WO01/64846)
[37] WO2006/071563
[38] WO2005/113758
[39] WO97/37001
[40] Bentolila et al. (1999) Mamm Genome 10(7):699-705.
[41] Dustin et al. (2010) Seminars in Cancer Biology; Vol. 20 (4): 234-245
[42] Zhang & Liu (2003) Expert Rev Mol Diagn 3:237-248.
[43] Hengen (1995) Trends Biochem Sci 20:372-373,
[44] Gennaro (2000) Remington: The Science and Practice of Pharmacy. 20th edition, ISBN: 0683306472
[45] Banzhoff (2000) Immunology Letters 71:91-96
[46] Nony et al. (2001) Vaccine 27:3645-51
[47] EP-B-0870508
[48] U.S. Pat. No. 5,948,410
[49] WO2007/052163
[50] WO2007/052061
[51] WO90/14837
[52] Podda & Del Giudice (2003) Expert Rev Vaccines 2:197-203
[53] Podda (2001) Vaccine 19: 2673-2680
[54] Vaccine Design: The Subunit and Adjuvant Approach (eds. Powell & Newman) Plenum Press 1995 (ISBN 0-306-44867-X)
[55] Vaccine Adjuvants: Preparation Methods and Research Protocols (Volume 42 of Methods in Molecular Medicine series). ISBN: 1-59259-083-7. Ed. O'Hagan
[56] Allison & Byars (1992) Res Immunol 143:519-25
[57] Hariharan et al. (1995) Cancer Res 55:3486-9
[58] US-2007/014805
[59] US-2007/0191314
[60] Suli et al. (2004) Vaccine 22(25-26):3464-9
[61] WO95/11700
[62] U.S. Pat. No. 6,080,725
[63] WO2005/097181
[64] WO2006/113373
[65] Potter & Oxford (1979) Br Med Bull 35: 69-75
[66] Greenbaum et al. (2004) Vaccine 22:2566-77
[67] Zurbriggen et al. (2003) Expert Rev Vaccines 2:295-304
[68] Piascik (2003) J Am Pharm Assoc (Wash D.C.). 43:728-30
[69] Mann et al. (2004) Vaccine 22:2425-9
[70] Halperin et al. (1979) Am J Public Health 69:1247-50
[71] Herbert et al. (1979) J Infect Dis 140:234-8
[72] Chen et al. (2003) Vaccine 21:2830-6
[73] Yang et al., (2010) Vaccine 28: 3308-3311

### SEQUENCES

SEQ ID NO: 1 (forward primer to amplify a fragment of LINE ORF2)

ACTGTAGTGAGAGATGAAGAGG

SEQ ID NO: 2 (reverse primer to amplify a fragment of LINE ORF2)

GGCGTATACCTGTTCATAAT

SEQ ID NO: 3 (2078 bp fragment of LINE-ORF2)

ACTGTAGTGAGAGATGAAGAGGGACACTATATCATACTTAAAGGATCTA
TCCAACAAGAGGACTTAACAATCCTCAATATATATGCCCCGAATGTGGG
AGCTGCCAAATATATAAATCAATTATTAACCAAAGTGAAGAAATACTTA
GATAATAATACACTTATACTTGGTGACTTCAATCTAGCTCTTTCTATAC
TCGATAGGTCTTCTAAGCACAACATCTCCAAAGAAACGAGAGCTTTAAA
TGATACACTGGACCAGATGGATTTCACAGATATCTACAGAACTTTACAT
CCAAACTCAACTGAATACACATTCTTCTCAAGTGCACATGGAACTTTCT
CCAGAATAGACCACATATTGGGTCACCAATCGGGTCTGAACCGATACCA
AAAGATTGGGATCGTCCCCTGCATATTCTCAGACCATAATGCCTTGAAA
TTAGAACTAAATCACAACAAGAAGTTTGGAAGGACCTCAAACACGTGGA
GGTTAAGGACCATCCTGCTAAAAGATGAAAGGGTCAACCAGGAAATTAA
GGAAGAATTAAAAAGATTCATGGAAACTAATGAGAATGAAGATACAACC
GTTCAAAATCTTTGGGATGCAGCAAAAGCAGTCCTGAGGGGGAAATACA
TCGCAATACAAGCATCCATTCAAAAACTGGAAAGAACTCAAATACAAAA
GCTAACCTTACACATAAAGGAGCTAGAGAAAAAACAGCAAATGGATCCT
ACACCCAGGAGAAGAAGGGAGTTAATAAAGATTCGAGCAGAACTCAACG
AAATCGAAACCAGAAGAACTGTGGAACAGATCAACAGAACCAGGAGTTG
GTTCTTTGAAAGAATTAATAAGATAGATAAACCATTAGCCAGCCTTCTT
AAAAAGAAGAGAGAGAAGACTCAAATTAATAAAATCATGAATGAGAAAG
GAGAGATCACTACCAACACCAAGGAAATACAAACGATTTTAAAAACATA
TTATGAACAGGTATACGCCAATAAATTAGGCAATCTAGAAGAAATGGAC
GCATTCCTGGAAAGCCACAAACTACCAAAACTGGAACAGGAAGAAATAG
AAAACCTGCACAGGCCAATAACCAGGGAGGAAATTGAAGCAGTCATCAA
AAACCTCCCAAGACACAAGAGTCCAGGGCCAGATGGCTTCCCAGGGGAA
TTTTATCAAACGTTTAAAGAAGAAATCATACCTATTCTCCTAAAGCTGT
TTGGAAAGATAGAAAGAGATGGAGTACTTCCAAATTCGTTTTATGAAGC
CAGCATCACCTTAATTCCAAAACCAGACAAAGACCCCACCAAAAAGGAG
AATTACAGACCAATATCCCTGATGAACATGGATGCAAAAATTCTCAACA
AGATACTGGCCAATAGGATCCAACAGTACATTAAGAAAATTATTCACCA
TGACCAAGTAGGATTTATCCCCGGGACACAAGGCTGGTTCAACACCCGT
AAAACAATCAATGTGATTCATCATATCAGCAAGAGAAAAACCAAGAACC
ATATGATCCTCTCATTAGATGCAGAGAAAGCATTTGACAAAATACAGCA
TCCATTCCTGATCAAAACTCTTCAGAGTGTAGGGATAGAGGGAACATTC
CTCGACATCTTAAAAGCCATCTACGAAAAGCCCACAGCAAATATCATTC
TCAATGGGGAAGCACTGGGAGCCTTTCCCCTAAGATCAGGAACAAGACA
GGGATGTCCACTCTCACCACTGCTATTCAACATAGTGGTGGAAGTCCTA
GCCTCAGCAATCAGACAACAAAAAGACTTTAGGGGCATTCAATTTGGCA
AAGAAGAAGTCAAACTCTCCCTCTTCGCCGATGAGATGATCCTCTACAT
AGAAAACCCAAAAGTCTCCACCCCAAGATTGCTACAACTCATGCAGCAT
TGTGGTAGCGTGGCAGGATACATCATCAATGCCCAGAAATCAGTGGCAT
TTCTATACACTAACAATGAGACTGAAGAAAGAGAAATTAAGGAGTCAAT
CCCATTTACAATTGCACCCAAAAGCATAAGATACCTAGGAATAAACCTA
ACCAGGGAGGTAAAGG

SEQ ID NO: 4 (central A-rich region of a SINE element)

AAAAATACATACATACATACATACATACA

1-54. (canceled)

55. A method for determining the presence of host cell DNA in a composition comprising a biological product produced in a host cell, comprising the steps of:
i) providing a sample of a composition comprising a biological product produced in a host cell;
ii) amplifying from the sample from step (i) at least a fragment of a repetitive element or of a housekeeping gene of the host cell;
iii) calculating a copy number of the repetitive element or the housekeeping gene using the amplified DNA from step (ii); and
iv) calculating based on the copy number of the repetitive element or the housekeeping gene from step (iii): (a) an amount of residual host cell DNA in the composition; (b) a DNA safety factor (SF) of the composition; (c) a ratio R of oncogenes to repetitive element or housekeeping gene in the composition; or any combination thereof.

56. The method of claim 55, wherein the DNA safety factor (SF) is determined by the formula:
$$SF = \frac{{N}_{critical}^{oncogenes}}{{N}_{dose}^{oncogenes}}$$
wherein N_{critical}^{oncogenes} is the maximum number of oncogenes per dose which may be present in a dose of the composition.

57. The method of claim 55, wherein a dose of the composition is assumed to comprise x ng of cellular DNA from the host cell and the safety factor SF is calculated by the formula:
$$SF = \frac{{N}_{critical}^{oncogenes}}{{c}_{onco} \times \frac{x ng}{{c}_{DNA}}}$$
wherein N_{critical}^{oncogenes} is the required number of oncogenes to produce a tumour, c_{onco} is the critical concentration of oncogenes in the monobulk [oncogenes/mL] and c_{DNA} is the concentration of the host cell DNA in the composition [ng/mL].

58. The method of claim 57, wherein the composition is assumed to comprise 10 ng of cellular DNA from the host cell.

59. The method of claim 55, wherein a dose of the composition is defined by its volume and the safety factor SF is calculated by the formula:
$$SF = \frac{{N}_{critical}^{oncogenes}}{{c}_{onco} \times {V}_{dose}}$$
wherein N_{critical}^{oncogenes} is the required number of oncogenes to produce a tumour, c_{onco} is the critical concentration of oncogenes in the monobulk [oncogenes/mL] and V_{dose} is the volume of a dose of the composition [mL].

60. The method of claim 55, wherein a dose of the composition is defined by the amount of the biological product and the safety factor SF is calculated by the formula:
$$SF = \frac{{N}_{critical}^{oncogenes}}{{c}_{onco} \times \frac{{c}_{dose}}{{c}_{actual}}}$$
wherein N_{critical}^{oncogene} is the required number of oncogenes to produce a tumour, c_{onco} is the concentration of oncogenes in the monobulk [oncogenes/mL], c_{dose} is the concentration of the active ingredient per dose [μg/dose] and c_{actual} is the actual concentration of the active ingredient in the composition [μg/mL].

61. The method of claim 55, wherein the ratio R of oncogenes to repetitive element or housekeeping gene is calculated by the formula
$$R = \frac{{N}_{onco} \times {c}_{DNA}}{{m}_{hap . Gen} \times {N}_{rep / mL}}$$
wherein N_{onco} is the number of oncogenes per genome; Nᵣₑₚ is the number of repetitive elements/housekeeping genes [rep/mL]; c_{DNA} is the concentration of the cell DNA in the test sample [pg/mL]; and, m_{hap.gen} is the mass of the haploid genome of the cell.

62. The method of claim 55, wherein the host cell is an eukaryotic cell that produces a virus.

63. A method for making a pharmaceutical composition, the method comprising the steps of:
i) calculating a safety factor (SF) of a sample of a composition comprising a biological product by at least one of the following formulae:
$$SF = \frac{{N}_{critical}^{oncogenes}}{{N}_{dose}^{oncogenes}}$$
wherein N_{critical}^{oncogenes} is the maximum number of oncogenes per dose which may be present in a dose of the composition;
$$SF = \frac{{N}_{critical}^{oncogenes}}{{c}_{onco} \times \frac{x ng}{{c}_{DNA}}}$$
wherein N_{critical}^{oncogenes} is the required number of oncogenes to produce a tumour, c_{onco} is the concentration of oncogenes in the monobulk [oncogenes/mL] and c_{DNA} is the concentration of the host cell DNA in the composition [ng/mL];
$$SF = \frac{{N}_{critical}^{oncogenes}}{{c}_{onco} \times {V}_{dose}}$$
wherein N_{critical}^{oncogenes} is the required number of oncogenes to produce a tumour, c_{onco} is the critical concentration of oncogenes in the monobulk [oncogenes/mL] and V_{dose} is the volume of a dose of the composition [mL]; and,
$$SF = \frac{{N}_{critical}^{oncogenes}}{{c}_{onco} \times \frac{{c}_{dose}}{{c}_{actual}}}$$
wherein N_{critical}^{oncogenes} is the required number of oncogenes to produce a tumour, c_{onco} is the critical concentration of oncogenes in the monobulk [oncogenes/mL], c_{dose} is the concentration of the active ingredient per dose [μg/dose] and c_{actual} is the actual concentration of the active ingredient in the composition [μg/mL]; and,
ii) if the calculated value of SF from step (i) is within an acceptable level, then, using the composition to formulate into a pharmaceutical composition.

64. The method of claim 63, wherein the acceptable level of SF is at least 10⁷.

65. The method of claim 63, wherein the pharmaceutical composition is a vaccine.

66. The method of claim 65, wherein the vaccine is an influenza vaccine.

67. The method of claim 65, wherein the vaccine is a live virus vaccine, inactivated virus vaccine, a whole virus vaccine, a split virus vaccine, or a viral subunit vaccine.

68. A composition comprising a biological product produced in a host cell, wherein the composition comprises fewer than n repetitive elements or housekeeping genes, wherein n is calculated by the formula:
$$n = \frac{320}{R} \frac{oncogenes}{mL}$$
wherein R is a ratio of oncogenes to repetitive elements or housekeeping genes in the host cell.
